(19) United States
(12) Patent Application Publication
(10) Pub. No.: US 2005/0256441 A1
(43) Pub. Date: Nov. 17, 2005

(54) Device for renal protection

(75) Inventors: Chaim Lotan, Jerusalem (IL); Teddy Weiss, Jerusalem (IL); David Meerkin, Ramat Beit-Shemesh (IL)

Correspondence Address: Martin MOYNIHAN;c/o ANTHONY CASTORINA, SUITE 207, 2001 JEFFERSON DAVIS HIGHWAY, ARLINGTON, VA, 22202, US

(73) Assignee: Hadasit Medical Research Services and Development; Ltd.

(21) Appl. No.: 10/831,227

(22) Filed: Apr. 26, 2004

(51) Int. Cl. A61M 1/00; A61M 37/00; A61M 29/00

(52) U.S. Cl. 604/4.01

(57) ABSTRACT

A device and method is provided for protecting the renal system from contrast dyes injected into the circulatory system during procedures such as angiograms and angioplasty. The device includes a catheter having a draining portion and an occluding portion. The device is inserted into the coronary sinus at a predetermined time, the occluding portion is inflated, and the contrast-injected blood is diverted. This blood may be reintroduced into the coronary arteries several times.

### FIELD AND BACKGROUND OF THE INVENTION

The present invention relates to a device and method for renal protection and, more particularly, to a device and method for prevention of renal damage due to the presence of contrast material.

Coronary artery disease (CAD) affects over 11 million people in the United States and results in over 19 million deaths yearly worldwide. CAD is usually diagnosed following chest pain, shortness of breath, or other typical symptoms. Several methods are commonly used for diagnosing CAD, including an electrocardiogram, blood tests, stress tests and angiograms.

Angiograms are considered the most accurate way to measure the degree and location of occlusions within the coronary arteries. To facilitate the angiogram, a catheter is inserted into an artery (typically the femoral artery), and advanced into the coronary arteries. A contrast material is then injected into the coronary arteries to depict blood flow and identify any blockages within the arteries.

Typically the contrast material is a radiographic dye, which can be viewed under X-ray. The material, although useful in diagnosing CAD, is harmful to the kidneys. Its nephrotoxic properties are well known, and it has been estimated that as many as 25% of angiogram procedures result in some renal impairment. The likelihood of impairment increases significantly in patients with previous kidney problems, and patients with repeat angiograms or angioplasty. This type of impairment, termed nephropathy, is associated with significant in-hospital and long-term morbidity and mortality.

U.S. Pat. No. 6,554,819 to Reich discloses a method and device for preventing contrast associated nephropathy. Specifically, when contrast solution is injected into the coronary artery of a patient, blood is prevented from flowing through the coronary sinus into the right atrium. The blood in the coronary sinus is bypassed to a filtration device which filters out the contrast solution from the blood and recirculates the blood back to the patient. Blood flow from the coronary sinus is blocked by a balloon catheter, introduced via the inferior vena cava, and expanded by a liquid.

Disadvantages of the device and method described therein include the need for a filtering system, and the possibility of a prolonged and more difficult procedure due to insertion of the catheter via the inferior vena cava, from which it is difficult to access the coronary sinus and due to the prolonged filling of the balloon (up to a few seconds). Particularly in a compliant vein, a prolonged filling of the balloon due to use of a liquid may cause the vein to expand, thus preventing complete blockage of the coronary sinus.

There is thus a widely recognized need for, and it would be highly advantageous to have, a system and method for preventing contrast-associated renal deficiencies, which is devoid of the above limitations.

### SUMMARY OF THE INVENTION

According to one aspect of the present invention there is provided a device for draining blood from a bodily vessel. The device includes a draining portion having a proximal end and a distal end, the draining portion having an outlet on the distal end and a tubing leading from the outlet to the proximal end, and an occluding portion, wherein the occluding portion is positioned proximal to the outlet, and is in fluid communication with a port for injecting gas to inflate the occluding portion.

According to further features in preferred embodiments of the invention described below, the tubing leads past the proximal end and outside of the device. According to yet further features, the occluding portion is a compliant blocking portion, such as an inflatable balloon. In different embodiments, the gas is carbon dioxide or helium.

According to a further aspect of the present invention there is provided a method for protecting the renal system from contrast dyes within the circulatory system. The method includes providing a catheter having a draining portion having a proximal end and a distal end, an outlet on the distal end and a tubing leading from the outlet to the proximal end and an occluding portion, wherein the occluding portion is positioned proximal to the outlet. The method further includes inserting the distal end of the draining portion into a predetermined location within a circulatory system, introducing an angiogram catheter into a coronary artery, injecting a contrast material into the coronary artery, thus providing contrast-injected blood, determining a moment of entry of the contrast-injected blood into the predetermined location, activating the occluding portion so as to block drainage of the contrast-injected blood beyond the predetermined location, diverting the contrast-injected blood into the outlet and through the tubing, determining an end of entry of the contrast-injected blood into the predetermined location, and deactivating the occluding portion so as to allow blood flow past the predetermined location to resume, wherein the deactivating occurs less than one second after the activating.

According to further features in preferred embodiments of the invention described below, the predetermined location is a coronary sinus location, such as a coronary sinus orifice or a branch of the coronary sinus. According to further features, the determining is done by timing, by examining an electrocardiogram, or by visualization. The activating includes introducing a gas into the occluding portion, wherein the occluding portion is expandable. In one embodiment, the gas is carbon dioxide. In another embodiment, the gas is helium. Alternatively, the occluding portion is an umbrella device and the activating includes expanding the umbrella device. In one embodiment, the inserting is done via the superior vena cava.

According to further features, the method further includes collecting the contrast-injected blood outside a body, and reinjecting the contrast-injected blood into the coronary arteries via the angiogram catheter. According to one embodiment, the method further includes repeating the reinjecting and the diverting and the collecting until viewing of the coronary arteries is completed, and removing the catheter from the circulatory system. According to yet another embodiment, the method further includes collecting the contrast-injected blood outside a body, separating the contrast material from the blood, and reinjecting the blood into the circulatory system.

According to yet another aspect of the present invention, there is provided a method for protecting the renal system from contrast dyes within the circulatory system, including providing a catheter having a draining portion and having a proximal end and a distal end, an outlet on the distal end and a tubing leading from the outlet to the proximal end, inserting the distal end of the draining portion into a coronary sinus location within a circulatory system, introducing an angiogram catheter into a coronary artery, injecting a contrast material into the coronary artery, thus providing contrast-injected blood, diverting the contrast-injected blood into the outlet and through the tubing, collecting the contrast-injected blood outside a body, reinjecting the contrast-injected blood into the coronary arteries via the angiogram catheter, repeating the reinjecting and diverting and collecting until viewing of the arteries is completed, and removing the draining portion from the coronary sinus location.

According to further features, the catheter further includes an occluding portion, and the method further includes determining a moment of entry of the contrast-injected blood into the coronary sinus location, activating the occluding portion so as to block drainage of the contrast-injected blood beyond the predetermined location, determining an end of entry of the contrast-injected blood into the predetermined location, and deactivating the occluding portion so as to allow blood flow past the predetermined location to resume.

According to further features, the activating includes introducing a fluid into the occluding portion, wherein the occluding portion is expandable and the fluid causes the occluding portion to expand. In one embodiment, the fluid is a liquid. In another embodiment, the fluid is a gas. In one embodiment, the occluding portion is a compliant balloon and the activating occurs rapidly by inflation of the compliant balloon. In another embodiment, the occluding portion is an umbrella device and the activating occurs by expanding the umbrella device.

According to yet another embodiment of the present invention, there is provided a method for protecting the renal system from contrast dyes within the circulatory system. The method includes providing a catheter having a draining portion having a proximal end and a distal end, an outlet on the distal end and a tubing leading from the outlet to the proximal end, inserting the distal end of the draining portion through the superior vena cava into a coronary sinus location within a circulatory system, introducing an angiogram catheter into a coronary artery, injecting a contrast material into the coronary artery, thus providing contrast-injected blood, diverting the contrast-injected blood into the outlet and through the tubing, and removing the draining portion from the coronary sinus location.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. In case of conflict, the patent specification, including definitions, will control. In addition, the materials, methods, and examples are illustrative only and not intended to be limiting.

### BRIEF DESCRIPTION OF THE DRAWINGS

The invention is herein described, by way of example only, with reference to the accompanying drawings. With specific reference now to the drawings in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

In the drawings:

FIG. 1 is an anterior cross-sectional view of the heart;

FIG. 2 is an anterior view of the heart showing the major coronary arteries supplying the heart tissue;

FIG. 3A is a posterior view of the heart, showing the coronary sinus;

FIG. 3B is cross-sectional superior view of the right atrium of the heart;

FIG. 4 is an anterior cross-sectional view of the heart depicting the relationship of the coronary sinus to the right atrium;

FIGS. 5A and 5B are elevational and cross-sectional views, respectively, of a catheter to be used in the present invention; and

FIGS. 6A-B are illustrations of a catheter for use in the present invention according to an alternative embodiment.

### DESCRIPTION OF THE PREFERRED EMBODIMENTS

The present invention is of an intracardiac device for renal protection. Specifically, the present invention provides a device and methods for decreasing the amount of contrast material processed by the renal system.

Before explaining at least one embodiment of the invention in detail, it is to be understood that the invention is not limited in its application to the details of construction and the arrangement of the components set forth in the following description or illustrated in the drawings. The invention is capable of other embodiments or of being practiced or carried out in various ways. Also, it is to be understood that the phraseology and terminology employed herein is for the purpose of description and should not be regarded as limiting.

The basic principle of the device and method disclosed herein is the capture and removal of the contrast-filled blood prior to entry into the renal system, and the recycling of the contrast material and/or the blood into the system for reuse. In a preferred embodiment, described more fully hereinbelow, the location of capture, or collection, is at the coronary sinus, prior to the blood entering the right atrium of the heart. Thus, although the device is described herein as an intercardiac device, it will be readily understood that a similar device designed to collect the material at any other suitable location within the circulatory system is contemplated as well.

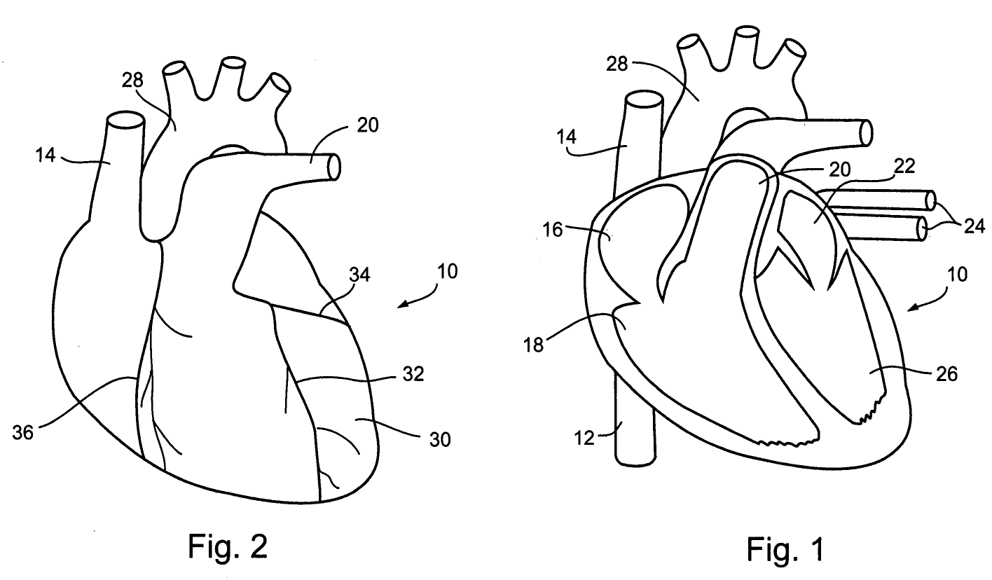

To better understand the operating principle of the preferred embodiment of the system of the present invention, a summary of the heart anatomy and circulation is provided, with reference to FIG. 1. FIG. 1 is an anterior cross-sectional view of the heart 10, illustrating the different chambers and the major blood vessels leading into and out of the chambers. The heart 10 is an organ that pumps blood throughout the body. Oxygen-poor blood enters the right side of the heart via the inferior vena cava 12 and the superior vena cava 14. The blood enters the right atrium 16, and is then pumped into the right ventricle 18, after which it exits the heart through the pulmonary artery 20. The blood becomes enriched with oxygen in the lungs, and the oxygenated blood then enters the left atrium 22 of the heart via one of the pulmonary veins 24. This blood is pumped to the left ventricle 26, after which it is pumped to the rest of the body via the aorta 28. The cycle then repeats itself.

The heart tissue itself needs a constant supply of oxygenated blood to enable it to pump the large quantities of blood necessary for the rest of the organs of the body. The heart receives oxygenated blood via the coronary arteries, which branch off from the beginning of the aorta. Reference is now made to FIG. 2, which is an anterior view of the heart showing the major coronary arteries supplying the heart tissue. The left side of the heart is supplied by the left main coronary artery 30, which branches off into the left anterior descending coronary artery 32 and the left circumflex coronary artery 34. The right side of the heart is supplied by the right coronary artery 36. The coronary arteries branch into progressively smaller vessels, which penetrate the heart muscle. If one of the coronary arteries is blocked, the cardiac muscle supplied by that artery will be affected, sometimes to the point of stopping cardiac function altogether.

In order to gather information regarding the state of the coronary arteries, very often a procedure known as an angiogram is performed, in which a contrast material is injected into the blood stream. Briefly, a catheter is inserted into the blood vessels with the aid of an imaging machine, such as an X-ray, that gives a real time image of the internal structures. The catheter is maneuvered into place, after which a contrast material is injected, and pictures are taken. The contrast material shows up on a radiographic image, which can give a clear picture of the status of the coronary arteries, thus enabling cardiologists to determine treatment plans with knowledge of the crucial facts regarding the arteries in question.

Unless removed, the dye will remain in the blood and will continue to circulate through the rest of the body along with the circulating blood. Under normal circumstances, the kidneys excrete the vast majority of the contrast material, an activity which can be damaging to the kidneys. The path of the circulating dye-injected blood follows the regular circulatory path. Most of the cardiac blood returning to the chambers of the heart flows through the coronary sinus, a venous structure that cuts across the posterior side of the heart, and drains directly into the right atrium. From there, the blood continues into the right ventricle, and out of the heart into the pulmonary system, after which it enters the left side of the heart via the pulmonary veins, exits the heart via the aorta, and eventually drains into various arteries, including the renal arteries. Thus, removal of the dyed blood for the purpose of preventing it from entering the renal system could theoretically be accomplished at any point from the coronary veins until it reaches the renal arteries. Optimally, if the blood is removed at an early stage along the process, a relatively high concentration of contrast material could be removed. Thus, in a preferred embodiment, removal of the blood is done prior to entry into the right atrium of the heart. A method and device are provided herein for removal of the blood at the coronary sinus orifice, just prior to flow into the right atrium. In an exemplary preferred embodiment, blood could be removed within specific branches of the coronary sinus, corresponding to specific injections of contrast into either the right or left coronary arteries.

It should be noted that although the preferred embodiments concern the coronary arteries, angiograms performed on other arteries within the body are included within the scope of the invention. For example, an angiography of the leg or arm will also result in contrast material that, if not removed, will circulate through the renal system. In these cases, removal of blood could be accomplished, for example, within the main femoral vein in a manner similar to the one described below for the coronary arteries.

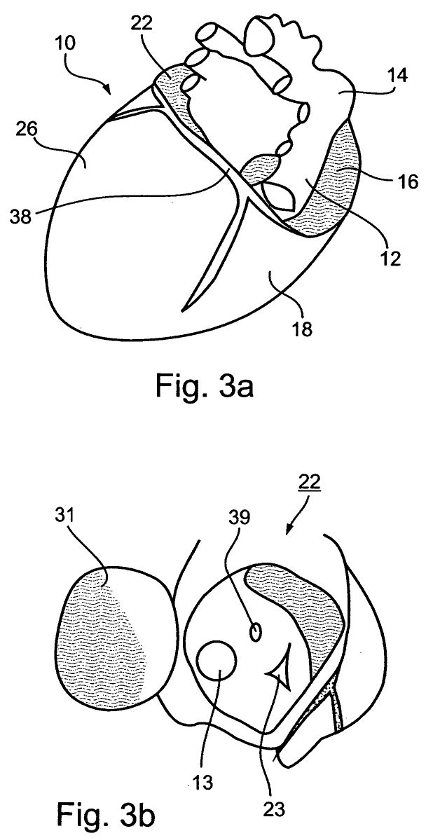

According to a preferred embodiment of the present invention, the target location for blood removal is the coronary sinus. The coronary sinus is the main drainage channel of venous blood from the myocardium. As shown in FIG. 3A, which is a posterior view of the heart, the coronary sinus 38 is situated within the atrioventricular groove on the posterior surface of the heart between the left atrium 22 and the left ventricle 26. Blood from the coronary sinus 38 drains into the right atrium 16 at the coronary sinus orifice. A cross-sectional superior view of the right atrium 22 is shown in FIG. 3B. FIG. 3B shows a cut-away flap 31 and the interior chamber of the right atrium 22. Three primary conduits are visible: the vena cava outlet 13, the coronary sinus orifice 39, and the tricuspid valve 23, which leads to the right ventricle 26. Blood collected at the coronary sinus orifice 39 is captured after it exits the cardiac tissue, before entering the general circulation.

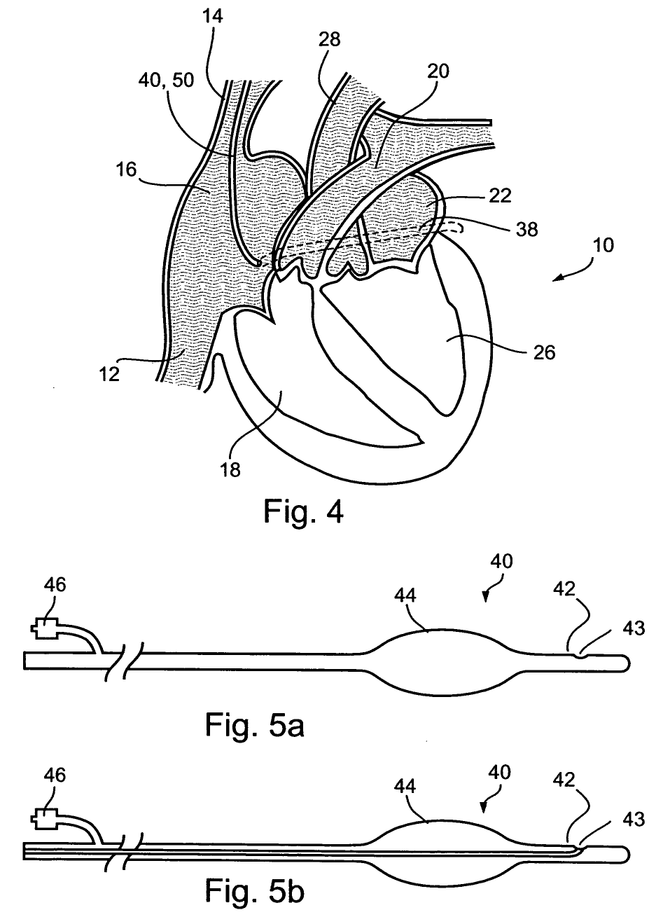

Reference is now made to FIG. 4, which is an anterior cross-sectional view of the heart, depicting the coronary sinus and its relationship to the right atrium. As shown in FIG. 4, a catheter 40, 50, inserted via the superior vena cava 14, is positioned within the coronary sinus 38, just at the orifice where the venous blood drains from the coronary sinus into the right atrium 16 of the heart 10. A tip of catheter 40, 50 is shown positioned within the coronary sinus. If blood flowing from the sinus is simultaneously blocked from entering the right atrium and diverted into catheter 40, 50, it is possible to collect the blood which has been injected with contrast material before it enters the general circulation.

Reference is now made to FIGS. 5A and 5B, which are elevational and cross-sectional views of a catheter 40 which can be used in the present invention. Catheter 40 has a draining portion 42 and an occluding portion 44. Draining portion 42 is located at a distal end of catheter 40, and includes an outlet 43 for collecting fluid. Occluding portion 44 is located proximal to draining portion 42, and is in fluid communication with a port 46 located at a proximal end of catheter 40. Catheter 40 may be a draining catheter, such as are commonly known in the art. For example, the Arrow Balloon wedge catheter, catalog numbers (5F) AI-07124 or (6F) AI-07126 (Arrow International, Reading, Pa., USA), can be used or adapted for use for the present application.

In a preferred embodiment, occluding portion 44 is a balloon or some other expandable material, which is caused to expand by introduction of fluid therein. Thus, expansion and deflation of occluding portion 44 is controlled by adjustment of the amount of fluid introduced at port 46. In an exemplary preferred embodiment, occluding portion 44 is a compliant type balloon, which can be expanded and deflated rapidly. For example, a sizing balloon (AMPLATZER® sizing balloon, AGA Medical Corporation, Golden Valley, Minn., USA) may be used. The type of fluid used to expand the balloon can vary. In one embodiment, the fluid is a liquid, such as water or saline. In a preferred embodiment, the fluid is a gas, preferably a dissolvable gas such as helium or carbon dioxide. An advantage to using a gas is the speed with which the balloon can be inflated, particularly if the balloon is made of a compliant material.

In an initial configuration, occluding portion 44 is deflated and thus, in close proximity or contacting the body of catheter 40. The relatively low initial profile allows the device to be inserted through the blood vessels on its way to the coronary sinus. Once the device is positioned in the appropriate location, fluid introduced through port 46 causes occluding portion 44 to expand, thus blocking the orifice from which blood can flow. Blood is then diverted, instead, through outlet 43 of catheter 40 and collected outside of the body.

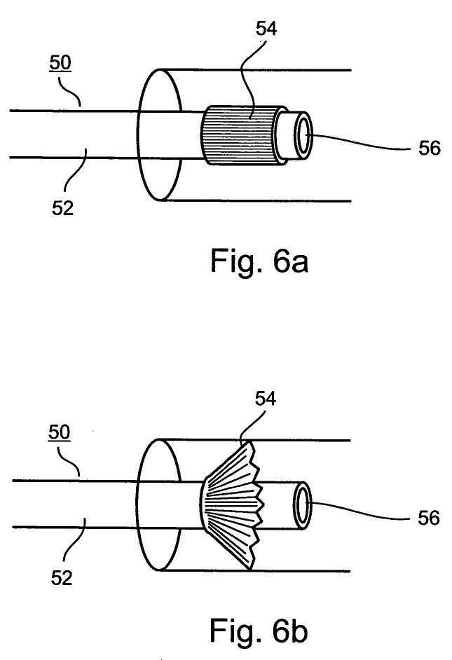

Reference is now made to FIGS. 6A-6B, which are illustrations of a catheter 50 in accordance with another embodiment of the present invention. The catheter 50 depicted in FIGS. 6A-6C is an umbrella catheter, which obviates the need for a balloon. Catheter 50, shown in FIG. 6A in an unexpanded state, includes a catheter body 52 with an occluding portion 54 positioned at a distal portion thereof. Occluding portion 54 is an umbrella configuration. In the unexpanded state, occluding portion 54 is substantially flush with catheter body 52. In an expanded state, as shown in FIG. 6B, occluding portion 54 is expanded to block blood flow. A distal tip of catheter 50 includes a draining portion 56, which can be for example, an outlet 53 for collection of blood blocked by occluding portion 54. In one embodiment, a suction mechanism is provided to enhance blood drainage from the vessel through catheter 50.

A method of draining contrast-injected blood from a vessel is provided herein. First, an angiogram catheter is introduced through the vessels and into the coronary arteries. Generally, the angiogram catheter is introduced through the femoral artery. Next, catheter 40, 50 of the present invention is positioned within the venous system via any one of several veins. The order of introducing the different catheters can be reversed, or they can be introduced simultaneously. In one embodiment, catheter 40, 50 is introduced via the jugular, subclavian, axillary or antecubital veins, and is then advanced through the superior vena cava and into the right atrium of the heart, until the tip of the catheter is just within the coronary sinus, as shown in FIG. 4. Alternatively, catheter 40, 50 is advanced through the femoral vein on either side, and through the inferior vena cava, and into the right atrium of the heart, until the tip of the catheter is just within the coronary sinus.

Once both catheters are in place, the angiogram procedure is started. Just prior to the time that the contrast-injected blood is about to enter the right atrium, occluding portion 44, 54 is expanded so as to block the drainage into the right atrium. Blood is then diverted through outlet 43, 53 into catheter 40, 50 and removed from the body. When the contrast-injected blood removal is completed, occluding portion 44, 54 is deflated. The timing of expansion and deflation of occluding portion 44 can be determined either by visualization on the X-ray, or by timing methods. In one embodiment, the expansion and deflation is based on the known time from when the contrast material is first injected to when the blood begins and ends flow into the right atrium. In yet another embodiment, an electrocardiogram could indicate at what point along the cycle the volume of relevant blood is located, and expansion and deflation times decided accordingly. Any one or combination of the above determinations could be made to most effectively remove the contrast-injected blood from the body. In a preferred embodiment, the total time of occlusion does not exceed a few seconds. Since veins have compliant properties, the longer the time of occlusion, the more of a chance the occlusion may be minimized due to expansion of the vessel. Thus, minimizing exposure time lessens this possibility, thus providing more efficient removal of the contrast material.

Once the blood is removed from the body, several possibilities exist. In a first embodiment, the removed blood/contrast is discarded, particularly in a case where a small number of injections are administered, such as in a diagnostic procedure. In another embodiment, the dye is separated from the blood, and the blood is reinfused into the patient. According to a preferred embodiment, the blood is reinjected via the angiogram catheter into the coronary arteries and reused as a contrast. Thus, if a practitioner desires multiple views of the coronary arteries within the same procedure period, the same contrast blood can be reused several times without the contrast entering the kidneys, or with only minimal amounts escaping the draining system and ultimately running through the renal system. Furthermore, the use of a filtering system may be eliminated. Once the procedure is completed, catheter 40, 50 is removed from the body.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims. All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention.

1. A device for draining blood from a bodily vessel, the device comprising:
a draining portion having a proximal end and a distal end, the draining portion comprises an outlet on said distal end and a tubing leading from said outlet to said proximal end; and
an occluding portion, wherein the occluding portion is positioned proximal to said outlet, said occluding portion in fluid communication with a port for injecting a gas to inflate said occluding portion.

2. The device of claim 1, wherein said occluding portion has compliant properties.

3. The device of claim 1, wherein said tubing leads past said proximal end and outside of said device.

4. The device of claim 1, wherein said occluding portion is an inflatable balloon.

5. The device of claim 1, wherein said gas is carbon dioxide.

6. The device of claim 1, wherein said gas is helium.

7. The device of claim 1, wherein said occluding portion is a compliant-type balloon.

8. A method for protecting the renal system from contrast dyes within the circulatory system, the method comprising:
providing a catheter having a draining portion comprising a proximal end and a distal end, an outlet on said distal end and a tubing leading from said outlet to said proximal end and an occluding portion, wherein the occluding portion is positioned proximal to said outlet;
inserting said distal end of said draining portion into a predetermined location within a circulatory system;
introducing an angiogram catheter into a coronary artery;
injecting a contrast material into said coronary artery, thus providing contrast-injected blood;
determining a moment of entry of said contrast-injected blood into said predetermined location;
activating said occluding portion so as to block drainage of said contrast-injected blood beyond said predetermined location;
diverting said contrast-injected blood into said outlet and through said tubing;
determining an end of entry of said contrast-injected blood into said predetermined location; and
deactivating said occluding portion so as to allow blood flow past said predetermined location to resume, wherein said deactivating occurs less than one second after said activating.

9. The method of claim 8, wherein said predetermined location is a coronary sinus location.

10. The method of claim 9, wherein said coronary sinus location is a coronary sinus orifice.

11. The method of claim 9, wherein said coronary sinus location is a branch of the coronary sinus.

12. The method of claim 8, wherein said determining is done by timing.

13. The method of claim 8, wherein said determining is done by examining an electrocardiogram.

14. The method of claim 8, wherein said determining is done by visualization.

15. The method of claim 8, wherein said activating includes introducing a gas into said occluding portion, and wherein said occluding portion is expandable and said gas causes said occluding portion to expand.

16. The method of claim 15, wherein said gas is carbon dioxide.

17. The method of claim 15, wherein said gas is helium.

18. The method of claim 15, wherein the occluding portion is an umbrella device and wherein said activating is done by expanding said umbrella device.

19. The method of claim 15, wherein said inserting is done via the superior vena cava.

20. The method of claim 8, further comprising:
collecting said contrast-injected blood outside a body; and
reinjecting said contrast-injected blood into the coronary arteries via said angiogram catheter.

21. The method of claim 20, further comprising:
repeating said reinjecting and said diverting and said collecting until viewing of the coronary arteries is completed; and
removing said catheter from the circulatory system.

22. The method of claim 8, further comprising:
collecting said contrast-injected blood outside a body;
separating said contrast material from said blood; and
reinjecting said blood into the circulatory system.

23. A method for protecting the renal system from contrast dyes within the circulatory system, the method comprising:
providing a catheter having a draining portion comprising a proximal end and a distal end, an outlet on said distal end and a tubing leading from said outlet to said proximal end;
inserting said distal end of said draining portion into a coronary sinus location within a circulatory system;
introducing an angiogram catheter into a coronary artery;
injecting a contrast material into said coronary artery, thus providing contrast-injected blood;
diverting said contrast-injected blood into said outlet and through said tubing;
collecting said contrast-injected blood outside a body;
reinjecting said contrast-injected blood into the coronary arteries via said angiogram catheter;
repeating said reinjecting and said diverting and said collecting until viewing of the coronary arteries is completed; and
removing said draining portion from said coronary sinus location.

24. The method of claim 23, wherein said catheter further comprises an occluding portion, and further comprising
determining a moment of entry of said contrast-injected blood into said coronary sinus location;
activating said occluding portion so as to block drainage of said contrast-injected blood beyond said predetermined location;
determining an end of entry of said contrast-injected blood into said predetermined location; and
deactivating said occluding portion so as to allow blood flow past said predetermined location to resume.

25. The method of claim 23, wherein said coronary sinus location is a coronary sinus orifice.

26. The method of claim 23, wherein said coronary sinus location is a branch of the coronary sinus.

27. The method of claim 24, wherein said determining is done by timing.

28. The method of claim 24, wherein said determining is done by examining an electrocardiogram.

29. The method of claim 24, wherein said determining is done by visualization.

30. The method of claim 24, wherein said activating includes introducing a fluid into said occluding portion, and wherein said occluding portion is expandable and said fluid causes said occluding portion to expand.

31. The method of claim 30, wherein said fluid is a liquid.

32. The method of claim 30, wherein said fluid is a gas.

33. The method of claim 24, wherein said occluding portion is a compliant balloon and wherein said activating occurs rapidly by inflation of said compliant balloon.

34. The method of claim 24, wherein the occluding portion is an umbrella device and wherein said activating is done by expanding said umbrella device.

35. The method of claim 23, wherein said inserting is done via the superior vena cava.

36. A method for protecting the renal system from contrast dyes within the circulatory system, the method comprising:
providing a catheter having a draining portion comprising a proximal end and a distal end, an outlet on said distal end and a tubing leading from said outlet to said proximal end;
inserting said distal end of said draining portion through the superior vena cava into a coronary sinus location within a circulatory system;
introducing an angiogram catheter into a coronary artery;
injecting a contrast material into said coronary artery, thus providing contrast-injected blood;
diverting said contrast-injected blood into said outlet and through said tubing; and
removing said draining portion from said coronary sinus location.

37. The method of claim 36, wherein said catheter further comprises an occluding portion, and further comprising
determining a moment of entry of said contrast-injected blood into said coronary sinus location;
activating said occluding portion so as to block drainage of said contrast-injected blood beyond said predetermined location;
determining an end of entry of said contrast-injected blood into said predetermined location; and
deactivating said occluding portion so as to allow blood flow past said predetermined location to resume.

38. The method of claim 36, wherein said coronary sinus location is a coronary sinus orifice.

39. The method of claim 36, wherein said coronary sinus location is a branch of the coronary sinus.

40. The method of claim 37, wherein said determining is done by timing.

41. The method of claim 37, wherein said determining is done by examining an electrocardiogram.

42. The method of claim 37, wherein said determining is done by visualization.

43. The method of claim 37, wherein said activating includes introducing a gas into said occluding portion, and wherein said occluding portion is expandable and said gas causes said occluding portion to expand.

44. The method of claim 43, wherein said gas is carbon dioxide.

45. The method of claim 43, wherein said gas is helium.

46. The method of claim 37, wherein said occluding portion is a compliant balloon and wherein said activating occurs rapidly by inflation of said compliant balloon.

47. The method of claim 37, wherein the occluding portion is an umbrella device and wherein said activating is done by expanding said umbrella device.

48. The method of claim 36, further comprising:
collecting said contrast-injected blood outside a body; and
reinjecting said contrast-injected blood into the coronary arteries via said angiogram catheter.

49. The method of claim 48, further comprising:
repeating said reinjecting and said diverting and said collecting until viewing of the coronary arteries is completed; and
removing said catheter from the circulatory system.

50. The method of claim 36, further comprising:
collecting said contrast-injected blood outside a body;
separating said contrast material from said blood; and
reinjecting said blood into the circulatory system.
